(19) United States
(12) Patent Application Publication
(10) Pub. No.: US 2009/0010891 A1
(43) Pub. Date: Jan. 8, 2009

(54) Sepsis Treatment Agent

(75) Inventor: Takashi Masuda, Tokyo (JP)

Correspondence Address: EDELL, SHAPIRO & FINNAN, LLC, 1901 RESEARCH BLVD., SUITE 400, ROCKVILLE, MD, 20850-3164, US

(73) Assignee: Toa Pharmaceutical Co., Ltd., Tokyo (JP)

(21) Appl. No.: 11/774,147

(22) Filed: Jul. 6, 2007

(51) Int. Cl. A61K 39/00; A61P 43/00

(52) U.S. Cl. 424/93.4

(57) ABSTRACT

To provide a bacterial translocation preventive for humans and animals comprising a probiotics formulation which has lactic acid bacteria, butyric acid bacteria, and saccharifying bacteria as the main components thereof. And to provide a sepsis treatment agent for humans and animals comprising a probiotics formulation which has lactic acid bacteria, butyric acid bacteria, and saccharifying bacteria as the main components thereof.

### BACKGROUND OF THE INVENTION

1. Field of the Invention

The present invention relates to a probiotics formulation which is effective in treatment of sepsis due to bacterial translocation. The probiotics formulation has lactic acid bacteria, butyric acid bacteria, and saccharifying bacteria.

2. Description of the Related Art

A large majority of infections develops from disease organisms entering the body from the outside. However, there are serious infections which do not seem likely to have come from outside. For example, in the intensive care unit, sepsis seen in the treatment of scalding or multiple traumas has an unknown source of infection. With this, the bacteria does not invade the body from outside, but the flora (mainly intestinal flora) of the patient is thought to be the pathogenic bacterial source of sepsis, and bacterial translocation results. In recent years, bacterial translocation is reported being seen even outside of intensive care units. The bacterial flora present inside the living body is seen as the infection source.

In sepsis, when infected with microorganisms such as bacteria or fungi, the infection is not localized, and the infection spreads throughout the body. Throughout the body, white blood cells such as macrophages and the like are activated excessively. The pathology of sepsis is from the dramatic inflammation reaction from the release of a large amount of inflammation factors. In very serious cases, multi-organ failure, and this becomes the source of fatality in intensive care units or neonatal ICUs.

In bacterial translocation, the bacteria normally present within the intestines for some reason passes through the intestinal walls and translocates to other organs and tissues (mesenteric lymph node, liver, kidney, spleen, intraperitoneal cavity, blood, and the like). This is the phenomenon in which bacteria and endotoxins inside the intestines passes through the intestinal epithelium wall. In animal experiments of bacterial translocation, using models for scalding, hemorrhagic shock, total parenteral nutrition, and the like, it is observed that intestinal bacteria (1) invade the lamina propria mucosae, (2) invade the intercellular space of the intestinal epithelium, and (3) invasion by macrophage phagocytosis.

Bacterial translocation is believed to occur clinically because of the following two phenomena. (1) over 30% or greater of patients with bacteremia in the intensive care unit do not have a clear infection source and much of the detected bacteria are intestinal gram negative flora. (2) in blood disorders, when cultured for monitoring purposes in patients with neutropenia, fecal bacteria are often detected in the blood.

Currently, there is no specific medicine for sepsis treatment. When sepsis occurs, for symptomatic treatment, systemic antibiotic administration and steroid administration is usual.

In the clinical setting, the frequency of sepsis due to bacterial translocation has increased greatly. From this standpoint, with the pathology described above, it is important to maintain a normal intestinal flora.

The object of the present invention is to prevent bacterial translocation and lower the frequency of sepsis by a probiotics formulation which is a mixture of lactic acid bacteria, butyric acid bacteria, and saccharifying bacteria.

### SUMMARY OF THE INVENTION

Lactic acid bacteria (Streptococcus faecalis T-110) (abbreviated as SF) used in the invention is a gram positive facultative anaerobic coccus isolated from normal adults, and lactic acid production is high, and it grows rapidly. Butyric acid bacteria (Clostridium butyricum TO-A) (abbreviated as CB) is a gram-positive obligate anaerobic bacillus which can form spores and is isolated from healthy adults. Butyric acid bacteria produces short-chain fatty acids such as butyric acid and acetic acid. In addition, saccharifying bacteria (Bacillus mesentericus TO-A) (abbreviated as BM) is a gram-positive aerobic bacillus which is isolated from soil and produces a starch hydrolyzing enzyme. These three bacteria form a symbiotic relationship. In other words, when there is a mix culture of SF and CB, the number of live bacteria of CB is approximately 10 times as that compared to the single culture. In addition, when SF and BM are cultured in starch culture, the number of live bacteria of SF increases approximately 100 fold. In addition, when confirming the suppressive action of SF and CB on pathogenic E. coli by anaerobic continuous-flow culture, a stronger suppressive action was seen when the symbiotic relationship between SF and CB was maintained as compared with SF or CB alone (Microbios Letters. 40.151-160, 1989). On the other hand, when BM and bifidus bacteria are mixed and cultured together, the growth rate of bifidus bacteria is increased 10-fold as compared with culturing bifidus bacteria by itself (Biomedical Letters. 48, 73-78, 1993). In this way, as is clear from the research results, the significance of mixing three types of bacteria has shown the proved effectiveness by the co-existence of these three types of bacteria.

The probiotics formulation which is a mixture of butyric acid bacteria, lactic acid bacteria, and saccharifying bacteria used in the present invention is an active live bacteria Bio3 (registered trademark) which was recognized in 1963. In addition, the Bio3 tablet was given approval for manufacture in 1974. For the mixture formulation of butyric acid bacteria, lactic acid bacteria, and saccharifying bacteria, the butyric acid bacteria, lactic acid bacteria, and saccharifying bacteria are cultured, and after completing culturing, the bacteria are collected by centrifugation, and this is mixed with a stabilizer and freeze dried. After drying, this is pulverized and mixed with a suitable base substance such as cornstarch, potato starch, dextrin, and the like. This formulation is constituted from butyric acid bacteria alone or from butyric acid bacteria, lactic acid bacteria, and saccharifying bacteria.

By their symbiotic action, lactic acid bacteria and butyric acid bacteria in the Bio3 can suppress the growth of bacteria such as salmonella and E. coli which is one of the bacteria causing sepsis (Seo G. et al.).

The present invention relates to a probiotics formulation that is effective in treatment of sepsis resulting from bacterial translocation. The probiotics formulation of the present invention comprises lactic acid bacteria, butyric acid bacteria, and saccharifying bacteria.

The present inventors have conducted intense study and showed the effectiveness of a probiotics formulation that is a mixture of lactic acid bacteria, butyric acid bacteria, and saccharifying bacteria for suppressing the growth of sepsis causing bacteria. The present invention was completed based on this finding.

In other words, the present invention relates to
1) A bacterial translocation preventive for humans and animals comprising a probiotics formulation which has lactic acid bacteria, butyric acid bacteria, and saccharifying bacteria as the main components thereof.
2) A sepsis treatment agent for humans and animals comprising a probiotics formulation which has lactic acid bacteria, butyric acid bacteria, and saccharifying bacteria as the main components thereof.

By oral administration of the probiotics formulation of the present invention, the progress of sepsis from bacterial translocation is suppressed or sepsis from bacterial translocation is prevented.

### DESCRIPTION OF THE PREFERRED EMBODIMENTS

The present invention is described in detail below.

The sepsis treatment agent of the present invention is a probiotics formulation.

The probiotics formulation of the present invention is a Bio3 (registered trademark), or a Bio3 tablet (registered trademark),

For the probiotics formulation of the present invention, butyric acid bacteria, lactic acid bacteria, and saccharifying bacteria are cultured. After completing culturing, the bacteria are collected by centrifugation, and a stabilizer is mixed, and this is freeze dried. After drying, this is mixed with a suitable base material of cornstarch, potato starch, dextrin, and the like. The probiotics formulation is constituted from butyric acid bacteria alone or from butyric acid bacteria, lactic acid bacteria, and saccharifying bacteria.

The present inventors discovered that administration of the probiotics formulation suppresses the growth of bacteria which causes sepsis and suppresses the progress of sepsis by suppressing the occurrence of bacterial translocation.

From the above, administration of the probiotics formulation suppresses or prevents the progress of sepsis. With these results, it is anticipated that the development of sepsis can be extended.

The sepsis treatment agent of the present invention is a probiotics formulation, or in other words, butyric acid bacteria, lactic acid bacteria, and saccharifying bacteria are mixed, and this is administered as a dosage form of preferably a powder, fine granule, granule, tablet, and the like.

The sepsis treatment agent of the present invention is a probiotics formulation of butyric acid bacteria, lactic acid bacteria, and saccharifying bacteria, or in other words, butyric acid bacteria, lactic acid bacteria, and saccharifying bacteria are mixed. When it is a powder, fine granule, or granule, the concentration is in the range of 10 mg to 1000 mg.

The sepsis treatment agent of the present invention suppresses bacterial translocation and is anticipated to treat or prevent sepsis.

### Embodiment 1

The results of the experiment when pathogenic E. coli infection models of Japanese white rabbits are given the live bacteria formulations of the three bacteria species mixture are shown below. There was a Bio3 administration group in which two day old Japanese white rabbits were given Bio3 with artificial milk and a control group which was not given Bio3. To these two groups, pathogenic E. coli K-1 strains were administered. Afterwards, the translocation of K-1 into each of the organs was studied. Four days after K-1 administration, the rabbits were killed. There were no differences in the spleen and liver, but in the mesentery lymph node, there was a significant suppression in the translocation of K-1 in the Bio3 administration group. In addition, in the histology of the intestinal mucosa, no mucosa disturbance was observed in the Bio3 group (Table 1).

TABLE 1
BT rate and histopathological observation of the young rabbits
given Bio3

| | BT rate in the | |
|---|---|---|
| | MLN (%) | Aplasia in MLN (%) |
| Control | 64.3 | 28.6 |
| BIO-THREE | 33.3 | 0 |

### Embodiment 2

The sepsis treatment agent was administered 3 times a day at 1 g each time using a live bacteria formulation of a mix of the three bacteria. The patients were hospitalized in a psychiatric hospital. The patients were blood culture positive which is a predictor for bacterial translocation. In 4 cases, imipenem/cilastatin (IPM/CS) was administered, and in 4 cases, latamoxef (LMOX) was given. During the observation period, CRP (C reactive protein) increased slightly, and E. coli and enterococcus and the like were detected by culturing blood. After observation for 1 year, two cases died from both groups, and two cases were cured.

Next, 5 cases were treated with meropenem and Bio3, and two cases were given simultaneously latamoxef and Bio3, and these were observed for 1 year. As a result, in both groups, no deaths were seen, and all cases were cured. As a result of autopsy, there were no findings that would suggest mechanical trauma such as bowel perforation or the like.

TABLE 2
The effect of probiotics of a mix of 3 bacteria species
in sepsis patients

| | Treatment course of sepsis | | |
|---|---|---|---|
| Drugs | Deaths* | Cured* | death rate (%) |
| Imipenem | 2 | 2 | 50 |
| Imipenem + BIO-THREE | 0 | 5 | 0 |
| Latamoxef | 2 | 2 | 50 |
| Latamoxef + BIO-THREE | 0 | 2 | 0 |

What is claimed is:

1. A bacterial translocation preventive for humans and animals comprising a probiotics formulation which has lactic acid bacteria, butyric acid bacteria, and saccharifying bacteria as the main components thereof.

2. A sepsis treatment agent for humans and animals comprising a probiotics formulation which has lactic acid bacteria, butyric acid bacteria, and saccharifying bacteria as the main components thereof.
